(19) United States
(12) Patent Application Publication
(10) Pub. No.: US 2006/0287669 A1
(43) Pub. Date: Dec. 21, 2006

(54) Catheter

(75) Inventors: Brendan Casey, Churchtown (IE); Avril O'Higgins, Renmore (IE); Eamon Brady, Elphin (IE); Patrick Griffin, Castlegar (IE); Michael Gilvarry, Ballina (IE)

Correspondence Address: RISSMAN JOBSE HENDRICKS & OLIVERIO, LLP, ONE STATE STREET, SUITE 800, BOSTON, MA, 02109, US

(73) Assignee: Salviac Limited, Dublin (IE)

(21) Appl. No.: 11/314,152

(22) Filed: Dec. 21, 2005

Related U.S. Application Data
(63) Continuation of application No. 10/123,514, filed on Apr. 17, 2002, now abandoned.

(30) Foreign Application Priority Data
Apr. 17, 2001 (IE) 2001/0376

(51) Int. Cl. A61M 29/00

(52) U.S. Cl. 606/200

(57) ABSTRACT

A retrieval catheter having a retrieval catheter tip 1 at a distal end thereof, the tip being suitable for retrieving an embolic protection filter into the tip 1. The tip 1 comprises a flexible tip body 2 and two stiff reinforcement columns 3. The tip body 2 is radially expandable to facilitate retrieval of a filter into the tip 1, and the columns 3 extend longitudinally along the tip body 2 to prevent buckling of the tip 1 upon retrieval of the filter into the tip. The cross-sectional area of the columns 3, the cross sectional area of the tip body 2, and the overall cross-sectional area of the tip 1 all remain substantially unchanged along the length of the tip 1. This ensures the reinforcement provided to the tip 1 by the columns 3 remains substantially uniform along the length of the tip 1. Thus no longitudinal squeezing of the filter as it is retrieved into the tip 1 will be caused.

This invention relates to a catheter, in particular it relates to a retrieval catheter tip for retrieving an article into the tip.

It is known to insert a retrieval catheter into a body passageway of a patient and to advance the retrieval catheter through the body passageway to retrieve an article, such as an embolic protection filter, from the passageway. During advancement of the retrieval catheter through the body passageway, the retrieval catheter tip must present a low profile to facilitate advancement through potentially narrow and/or tortuous passageways, and also to avoid causing damage or discomfort to the patient.

Conventional retrieval catheters suffer from a number of disadvantages. Generally the article to be retrieved from the body has a larger size than the retrieval catheter tip. It is therefore difficult to retrieve the relatively large article into the relatively small retrieval catheter tip. In addition, retrieval of the large article into the retrieval catheter tip is prone to cause buckling of the retrieval catheter tip due to the large compressive forces acting on the tip during the retrieval process.

U.S. Pat. No. 5,312,417 describes a laparoscopic cannula comprising a rigid tubular member with an insufflation port component at a proximal end and an expandable receiver portion at a distal end. The receiver portion includes an elastic or pleated web which is provided along an inner surface with a plurality of longitudinally extending resilient ribs. The ribs are said to have an internal spring bias to maintain the ribs in a normally straightened configuration. When a body organ is pulled into the distal end of the receiver the ribs are said to expand outwardly to permit the severed organ to be pulled into the web by a laparoscopic instrument.

One of the problems with a cannula receiving portion of this type is that there is a tendency of the tip to buckle as an organ is pulled into the web due to the large frictional forces acting on the tip during the retrieval process. This is particularly the case with organs which are large relative to the distal tip. This can lead to a loss or at least a reduction in control over the retrieval process with the possible risk of the organ to be retrieved not being held sufficiently tightly in the distal tip.

In the case of laparoscopic surgery in the abdomen of the patient the catheter has a relatively large outside diameter of typically 5 mm to 10 mm. Thus, even if some control of the organ is lost it can be manipulated so that a sufficient amount of the organ is held in the distal tip to facilitate retrieval through the relatively large incision in a patient's abdomen. In the event that the organ is not retrieved a surgeon can readily re-position the distal receiving portion and manipulate the organ again using a laparoscopic instrument. If necessary, the organ can be cut into a number of parts which can be more easily retrieved. The loss of control of an organ part is rarely a major complication in such laparoscopic procedures.

This is in complete contrast to retrieval of an embolic protection filter which is deployed in a patient's artery distally of a treatment site to capture emboli which may become dislodged from the patient's artery during an interventional procedure such as an angioplasty and/or stenting. Such emboli can have major life threatening consequences to a patient if they are not completely and safely captured. They could travel to the brain causing a stroke.

On completion of the interventional procedure it is necessary to retrieve the filter containing the captured emboli. The embolic load captured by the filter can be large and the retrieval of such filters is therefore a major and potentially dangerous procedure. The retrieval of the filter represents an occasion in which emboli could potentially be released from the filter.

This invention is aimed at overcoming at least some of the problems associated with known retrieval catheter tips. In particular this invention is directly towards providing a retrieval catheter tip into which an embolic protection filter or the like may be safely retrieved.

### STATEMENTS OF INVENTION

According to the invention, there is provided a retrieval catheter tip for retrieving an article into the tip, the tip comprising:—

an expandable tip body for retrieving an article into the tip;

the tip body being resilient to retain a retrieved article within the tip; and

a reinforcement column extending along the tip body;

the column being configured to provide substantially uniform reinforcement to the tip substantially along the length of the column.

In the retrieval catheter tip of the invention, the tip body is expandable to facilitate retrieval of an article, such as an embolic protection filter, into the tip. The resilient nature of the tip body then safely retains the retrieved article within the tip. In this manner the invention provides a simple and compact arrangement for retrieving and safely retaining an article, such as an embolic protection filter, from a body passageway.

The reinforcement columns provide the tip with longitudinal reinforcement to prevent kinking of the tip body during the retrieval process.

Longitudinal squeezing of an article being retrieved could result in some or all of the article not being safely retained within the tip. In the retrieval catheter tip of the invention, the columns provide substantially uniform reinforcement along the length of the tip. In this manner, the tip ensures that no longitudinal squeezing or extruding of the article being retrieved will occur during the retrieval process.

In one case, the ratio of the circumferential dimension of the column to the circumferential dimension of the tip body is substantially constant along the length of the tip.

In another case, the ratio of the wall thickness of the column to the wall thickness of the tip body is substantially constant along the length of the tip.

In a further case, the ratio of the cross-sectional area of the column to the cross-sectional area of the tip body is substantially constant along the length of the tip.

This is preferred because the tip can have a constant ratio of hard and soft material which provides good transition, good tip expansion and compressive resistance.

In one embodiment of the invention the column is substantially arcuate in cross-section. Preferably the angular extension of the column around the circumference of the tip is in the range of from 10 degrees to 145 degrees. Most preferably, the angular extension of the column around the circumference of the tip is in the range of from 105 degrees to 145 degrees. Ideally the angular extension of the column around the circumference of the tip is approximately 126 degrees.

In another preferred case the wall thickness of the tip at the location of the column is greater than or equal to the wall thickness of the tip body. Ideally the wall thickness of the tip at the location of the column is between 1 and 10 times the wall thickness of the tip body. Most preferably the wall thickness of the tip at the location of the column is two times the wall thickness of the tip body.

The ratio of the cross-sectional area of the column to the circumferential dimension of the column may be greater than or equal to the ratio of the cross-sectional area of the tip body to the circumferential dimension of the tip body. Preferably the ratio of the cross-sectional area of the column to the circumferential dimension of the column is between 1 and 10 times the ratio of the cross-sectional area of the tip body to the circumferential dimension of the tip body. Ideally the ratio of the cross-sectional area of the column to the circumferential dimension of the column is two times the ratio of the cross-sectional area of the tip body to the circumferential dimension of the tip body.

In one embodiment the column extends along substantially the full length of the tip body.

In another embodiment the column extends along part of the length of the tip body. Preferably the tip body extends distally of the column.

In a preferred embodiment the tip is of a length in the range of from 3 mm to 20 mm. Ideally the tip is of a length of approximately 13 mm.

In one case the tip at least partially tapers distally inwardly. In another case the tip at least partially tapers distally outwardly.

The wall thickness of the column and/or of the tip body may vary along at least part of the length of the tip. The wall thickness of the column and/or of the tip body may be substantially constant along at least part of the length of the tip.

In a preferred embodiment the tip is shaped for a smooth crossing profile. Ideally a distal end of the tip is rounded. Most preferably a distal end of the tip tapers distally inwardly.

The tip may comprise two or more columns spaced apart circumferentially around the tip. Ideally the tip comprises two columns. Most preferably the columns are substantially equi-spaced apart.

In one embodiment of the invention the tip body is integrally formed with the column to form the tip. Preferably the tip body and the column are co-extruded to form the tip.

In another embodiment the column is fixed to the pre-formed tip body to form the tip.

In a further embodiment the tip body and the column are injection moulded to form the tip.

The column may be at least partially embedded within the tip body. The column may be provided at least partially on an external and/or internal surface of the tip body.

Preferably the tip comprises bonding means between the column and the tip body. The column may be shaped to define a relatively large surface area between the column and the tip body. Preferably the bonding means comprises mechanical and/or chemical adhesion means.

In one preferred case the column and the tip body are at least partially of materials selected from the same groups of materials. The column and the tip body may be at least partially of materials selected from the peba group of materials. Preferably the column is of a material with a higher nylon concentration than the material of the tip body. The column and the tip body may be at least partially of materials selected from the fluoropolymer group of materials. Preferably the column is of a solid fluoropolymer and the tip body is of an expanded fluoropolymer. The column and the tip body may be at least partially of materials selected from the polyurethane group of materials. Preferably the column is of a material with a higher isocyanate concentration than the material of the tip body. The column and the tip body may be at least partially of materials selected from the polyester group of materials.

In another case the column and the tip body are at least partially of materials selected from different groups of materials. Preferably the column material is of a hardness at least 20 D greater than the tip body material. The column material may be a metal, such as stainless steel.

In a further embodiment the column and/or the tip body is of a composite material.

Desirably the tip comprises a hydrophilic coating.

In a preferred embodiment the tip comprises means to ease relative movement of the tip. The means may comprise at least one protrusion. Preferably the protrusion extends at least partially radially outwardly of the tip body. The protrusion may extend at least partially radially inwardly of the tip body. Ideally the protrusion extends longitudinally along at least part of the tip. The protrusion may be provided by the column.

In one case the tip comprises a low friction coating.

In a particularly preferred embodiment the tip is an embolic protection filter retrieval catheter tip.

The retrieval catheter tip of the invention is particularly suitable for use as a retrieval catheter tip for retrieving an embolic protection filter with captured embolic material therein from a vasculature. It is very important that the captured embolic material is not dislodged from within the filter during the retrieval process to prevent the embolic material from being extruded out of the filter and escaping back into the vasculature which could potentially lead to life-threatening consequences. Because the columns provide uniform reinforcement along the length of the tip, the filter will not be longitudinally squeezed during the retrieval procedure. Thus the possibility of embolic material being released back into the vasculature during retrieval using the retrieval catheter tip of the invention is minimised.

In another aspect the invention provides a retrieval catheter comprising a catheter shaft and a retrieval catheter tip of the invention at a distal end of the catheter shaft.

Preferably the tip is fixed to the shaft.

The column may extend proximally of the tip at least partially along the catheter shaft.

In one preferred case the catheter comprises means to centre the catheter during advancement. The centring means may comprise a centring catheter for protruding distally of the retrieval catheter tip. Preferably the centring catheter when protruding distally of the retrieval catheter tip forms a smooth transition to the retrieval catheter tip. Ideally the tip tapers distally inwardly. The centring catheter may taper proximally outwardly. The centring catheter is preferably retractable relative to the retrieval catheter tip. The centring catheter may have a tip shaped for a smooth crossing profile. Ideally the centring catheter tip is arrow-head shaped, or rounded, or ball-nose shaped.

According to a further aspect of the invention, there is provided a retrieval catheter tip assembly comprising a retrieval catheter tip of the invention and a lead-in catheter section at a distal end of the tip.

The lead-in section may taper distally inwardly.

The wall thickness of the lead-in section preferably varies along at least part of the length of the lead-in section.

In one case the lead-in section is fixed to the tip.

In another case the lead-in section is integral with the tip. Ideally the lead-in section is provided as a distal extension of the tip body.

### BRIEF DESCRIPTION OF THE DRAWINGS

The invention will be more clearly understood from the following description of some embodiments thereof, given by way of example only, with reference to the accompanying drawings, in which:—

FIG. 1 is a perspective view of a retrieval catheter tip according to the invention;

FIG. 2 is an end view of the tip of FIG. 1;

FIG. 2(a) to 2(c) are schematic views illustrating the tip of FIG. 1 in use;

FIG. 2(d) is a view along line I-I in FIG. 2;

FIG. 2(e) is a view along line II-II in FIG. 2;

FIG. 3 is a perspective view of another retrieval catheter tip according to the invention;

FIG. 4 is an end view of the tip of FIG. 3;

FIG. 5(a) is an end view of the tip of FIG. 1;

FIG. 5(b) is an end view of another retrieval catheter tip according to the invention;

FIG. 5(c) is an end view of the tip of FIG. 1;

FIGS. 5(d) to 5(j) are end views of other retrieval catheter tips according to the invention;

FIGS. 5(k) and 5(m) are stress-strain curves for tip bodies of other retrieval catheter tips according to the invention;

FIG. 5(n) is a graph illustrating the variation of percentage strain on the tip body with the percentage of the column in the circumference of the tip;

FIG. 6 is a perspective view of another retrieval catheter tip according to the invention;

FIG. 7 is an end view of the tip of FIG. 6;

FIG. 8 is a perspective view of a further retrieval catheter tip according to the invention;

FIG. 9 is an end view of the tip of FIG. 8;

FIG. 10 is an enlarged view of part of the tip of FIG. 9;

FIGS. 11 to 22 are views similar to FIGS. 8 to 10 of other retrieval catheter tips according to the invention;

FIGS. 22(a) to 22(d) are end views of other retrieval catheter tips according to the invention;

FIG. 23 is a side, cross-sectional view of the tip of FIG. 1;

FIGS. 24 to 27 are side, cross-sectional views of other retrieval catheter tips according to the invention;

FIGS. 28 and 29 are perspective, partially cut-away views of other retrieval catheter tips according to the invention;

FIGS. 30 to 32 are side, cross-sectional views of other retrieval catheter tips according to the invention;

FIG. 33(a) is a side, cross-sectional view of another retrieval catheter tip according to the invention;

FIG. 33(b) is a perspective, partially cut-away view of the tip of FIG. 33(a) in use; and

FIGS. 34(a) to 35(b) are views similar to FIGS. 33(a) and (b) of other retrieval catheter tips according to the invention.

### DETAILED DESCRIPTION

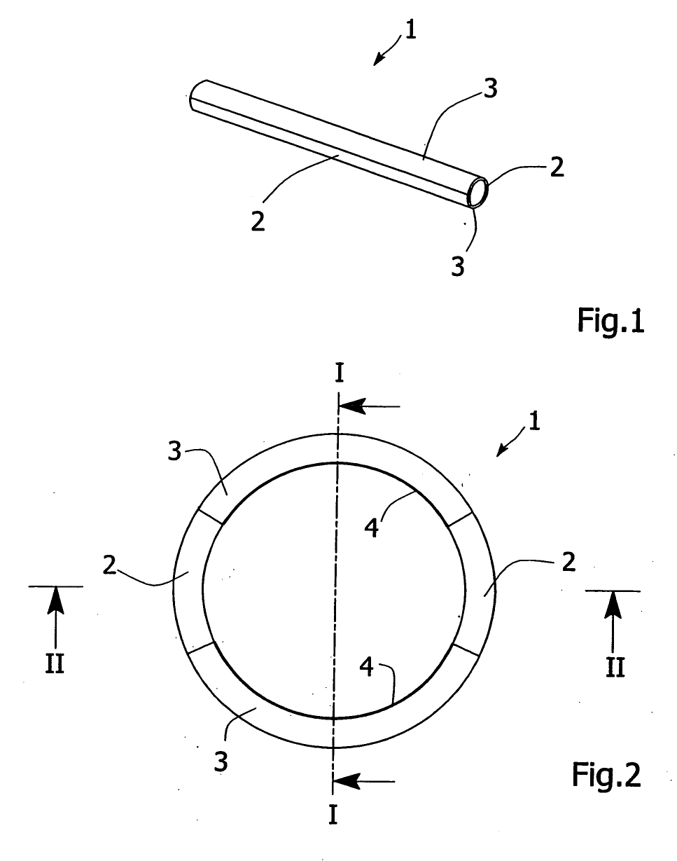

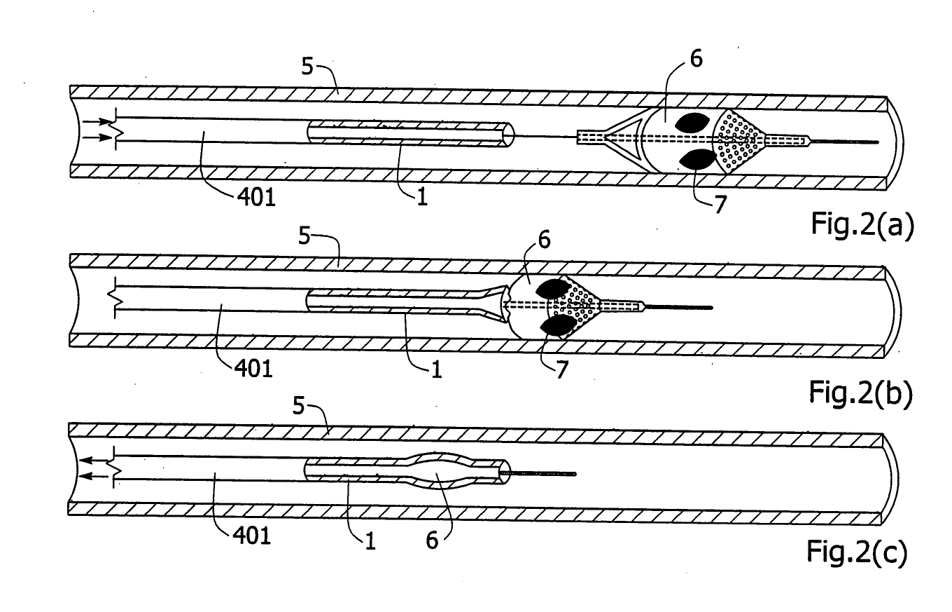

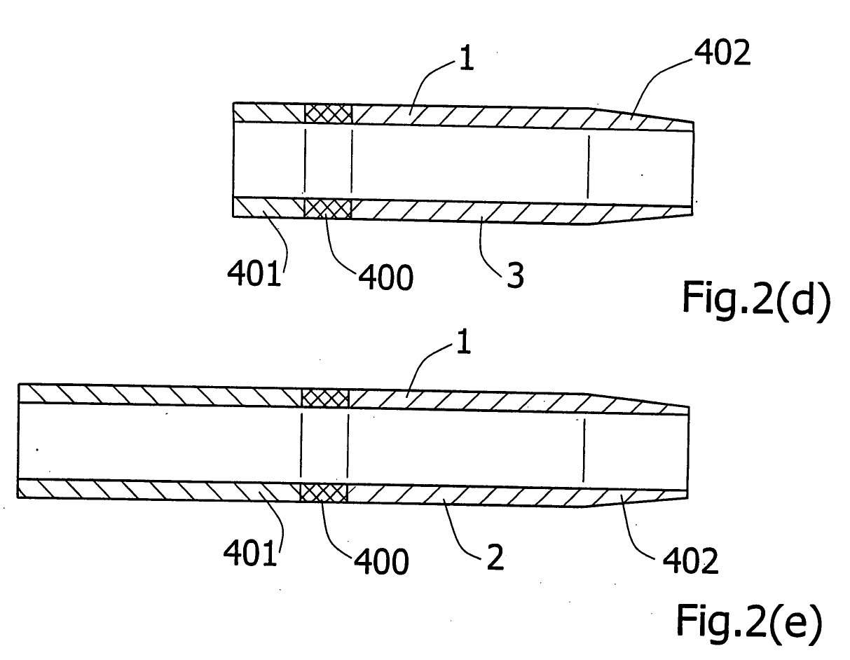

Referring to FIGS. 1 and 2 there is illustrated a retrieval catheter tip 1 according to the invention for retrieving an article, such as an embolic protection filter, into the tip 1. The tip 1 is provided in use at a distal end 400 of a retrieval catheter shaft 401, the tip 1 being fixed to the shaft 401 at the distal end 400 by any suitable means, such as by welding (FIGS. 2(d) and 2(e)).

A lead-in section 402 is provided distally of the tip 1. As illustrated in FIGS. 2(d) and 2(e), the wall thickness of the lead-in 402 decreases distally such that the lead-in 402 tapers distally inwardly. In this way, the lead-in 402 provides a smooth transition for passage through a vasculature or other body passageway, while permitting ease of retrieval of an article into the tip 1. In this case, the lead-in section 402 is provided as an integral extension of the tip 1. However in another case the lead-in section may be provided as a separate component from the tip and fixed to the tip by any suitable means. In such a case, the material of the lead-in section may be different from the materials of the tip.

The tip 1 comprises a flexible tip body 2, preferably of Tecothane from Thermedics, and at least one, and in this case two, stiff reinforcement columns 3, preferably of Pellethane from Dow. The tip body 2 is radially expandable in use to facilitate retrieval of an article into the tip 1, and the tip body 2 is also resilient to retain the article within the tip 1 once retrieved. The columns 3 prevent buckling of the tip 1, and extend longitudinally along the tip body 2.

In this case, the outer surface of the tip body 2 is flush with the outer surface of the columns 3, as illustrated in FIG. 2. Two thin layers 4 of tip body material extend circumferentially along the inner surface of the two columns 3 to link the two larger wings of the tip body 2 (FIG. 2). The columns 3 are equi-spaced apart circumferentially around the tip body 2, as illustrated in FIG. 2. In this way, the possibility of circumferential buckling of the tip 1 is minimised.

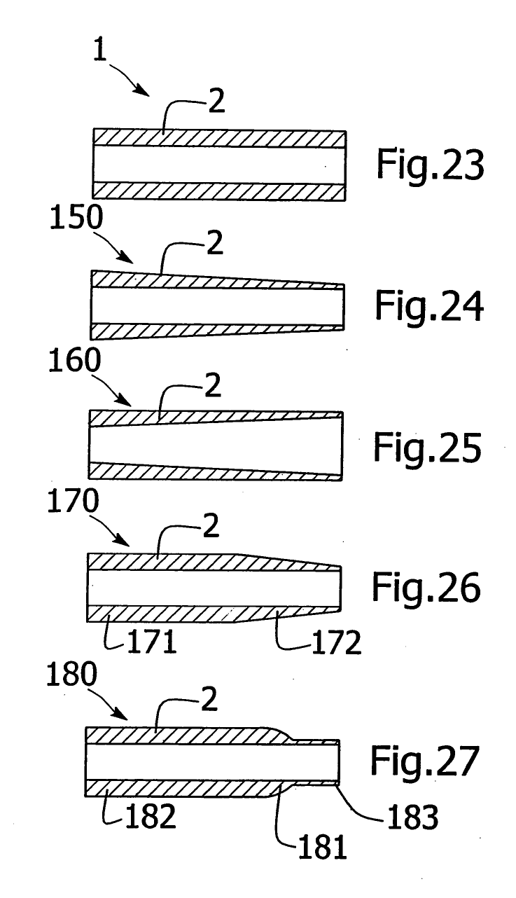

The cross-sectional area of the columns 3, the cross sectional area of the tip body 2, and the overall cross-sectional area of the tip 1 preferably all remain substantially unchanged along the length of the tip 1, as illustrated in FIG. 23. This configuration ensures the reinforcement provided to the tip 1 by the columns 3 remains substantially uniform along the length of the tip 1. Thus no longitudinal squeezing of the article being retrieved into the tip 1, such as an embolic protection filter, will be caused. In this manner the tip 1 will safely retain the retrieved article within the tip 1. In addition, the likelihood of buckling of the tip 1 during retrieval of an article into the tip 1 is minimised due to the presence of the reinforcement columns 3. This constant, cross-sectional area tip 1 may be easily and inexpensively manufactured by extrusion.

The cross section of the columns 3 is shaped to provide a high second moment of area relative to a plane of bending running parallel to the tip 1 through the central axis of the catheter, and the columns 3 are of a stiff material. In this way the critical buckling load for the tip 1 is maximised.

The tip body 2 is integrally formed with the columns 3 to form the tip 1 by, for example, a co-extrusion process. It is easier to form the tip 1 in the co-extrusion process with the thin layers 4 of tip body material along the inner surface of the columns 3. Large quantities of the tip 1 can be produced using co-extrusion, and the profile of the tip 1 formed can be easily controlled. Also because the materials are melted simultaneously, a secure bond between the reinforcement columns 3 may be readily achieved.

The tip body 2 and the columns 3 are shaped to define a large surface area between the tip body 2 and the columns 3 for secure bonding of the tip body 2 with the columns 3. Also the materials for the tip body 2 and the columns 3 are chosen to be suitably compatible for secure bonding of the tip body 2 with the columns 3.

The tip body material and the column material are preferably both selected from the polyurethane group of materials. Different grades of hardness of polyurethane are readily available for both the tip body and the reinforcement column. For example, the tip body material may have a hardness less than the range of 50D to 65D, and the column material may have a hardness greater than the range of 50D to 65D. Polyurethane materials can be easily co-extruded to form the tip 1. In addition, polyurethane is also a suitable material for welding, for example to fix the tip 1 to the distal end 400 of the retrieval catheter shaft 401.

The tip body material and the column material may alternatively both be selected from the peba group of materials. Different grades of hardness of peba are readily available for both the tip body and the reinforcement column. In one case, the tip body material preferably has a high ether concentration, a low nylon concentration and a hardness less than the range of 50 D to 65 D, and the column material preferably has a high nylon concentration, a low ether concentration and a hardness greater than the range of 50 D to 65 D. Peba materials can be easily co-extruded to form the tip 1. However peba materials have higher frictional properties, especially in the softer grades of peba. In addition peba materials are less suitable for welding, and thus fixing the tip 1 to the distal end 400 of the retrieval catheter shaft 401 by welding would be more difficult.

Alternatively, the tip body material and the column material may both be selected from the fluoropolymer group of materials, for example, a polytetrafluoroethylene. In such a case, the tip body material may be an expanded fluoropolymer, for example expanded polytetrafluoroethylene, and the column material may be a solid fluoropolymer.

Alternatively, the tip body material and the column material may both be selected from the polyester group of materials. In such a case, the tip body material will typically have a hardness less than the range of 50 D to 65 D, and the column material will typically have a hardness greater than the range of 50 D to 65 D.

It will be appreciated that a mixture/blend/compound of any of the above materials could alternatively be used for the tip body and/or the columns.

The column material may alternatively be chosen from a substantially different material to the tip body material. In this case, the tip body may be configured to thermally contract during manufacture to provide a compressive mechanical grip of the column. This may be achieved by co-extrusion, over extrusion or over moulding.

Further, the column 3 may be a two-layer construction. In this case the outer layer forms a tie layer between the inner column material and the flexible tip body.

In general, the actual material hardness used depends on the device profile and the retrieval forces. It is a general objective of the invention to render the tip trackable. To achieve this objective the hardness of the materials used is reduced. The tip body material is typically 15 D softer than the column material. Ideally the tip body material is more than 25 D softer than the column material.

A hydrophilic coating is usually provided around the exterior and/or interior of the retrieval catheter distal tip 1. This results in at least reduction and in some cases substantial elimination of platelet adhesion and fibrin build-up which could otherwise at least partially occlude the tip 1 and/or create a harmful thrombus.

A low friction coating may be provided around the exterior and/or interior of the tip 1 to reduce the insertion force required when advancing the tip 1 through a guide catheter and/or a body passageway. A low friction coating also reduces the retrieval force required for retrieval of an article into the tip 1.

The columns 3 may extend longitudinally proximally of the distal tip 1 along a part of or the entire length of the retrieval catheter. In this manner part or all of the retrieval catheter is longitudinally reinforced to prevent buckling of the retrieval catheter during advancement of the retrieval catheter, and/or during retrieval of an article into the tip 1.

In use, the retrieval catheter 401 is inserted into a body passageway, in this case a vasculature 5, and advanced through the vasculature 5 until the tip 1 is proximally adjacent of an article to be retrieved, such as an embolic protection filter 6 with captured embolic material 7 therein (FIG. 2(a)).

The tip 1 is then moved distally relative to the filter 6 to retrieve the filter 6 with the captured embolic material 7 into the tip 1 (FIG. 2(b)). The tip body 2 expands radially outwardly to accommodate the relatively large embolic protection filter 6 into the tip 1 during retrieval. No buckling of the tip 1 occurs during retrieval due to the presence of the reinforcement columns 3. In addition, because the reinforcement is substantially uniform along the length of the tip 1, no longitudinal squeezing or extruding of the filter 6 occurs during retrieval, and thus no extrusion of embolic material 7 out of the filter 6 occurs.

When the filter 6 has been fully retrieved into the tip 1 (FIG. 2(c)), the retrieval catheter 401 is withdrawn from the vasculature 5. The resilient nature of the tip body 2 ensures the embolic protection filter 6 and the captured embolic material 7 are safely retained within the tip 1 during retrieval and withdrawal.

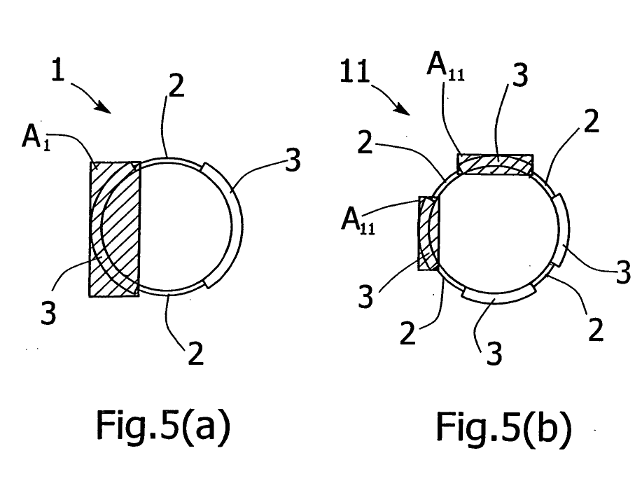

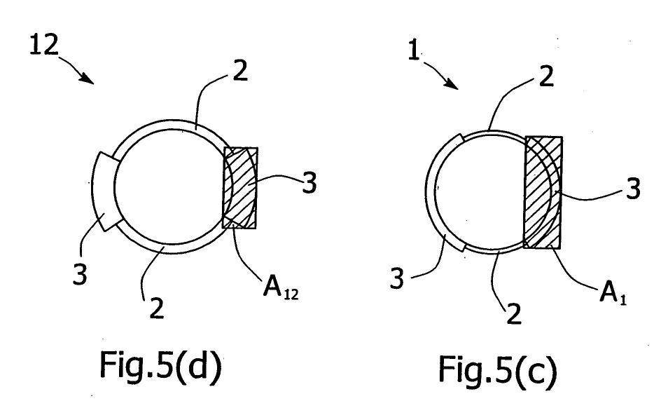

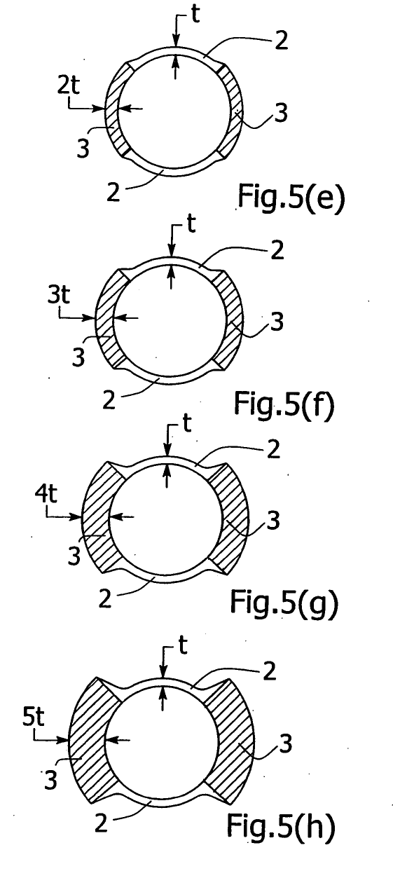

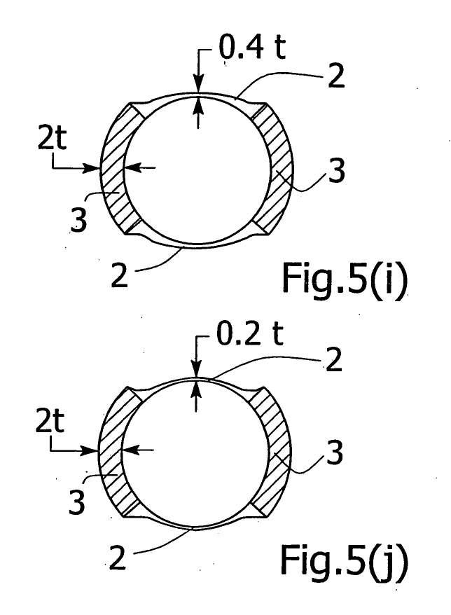

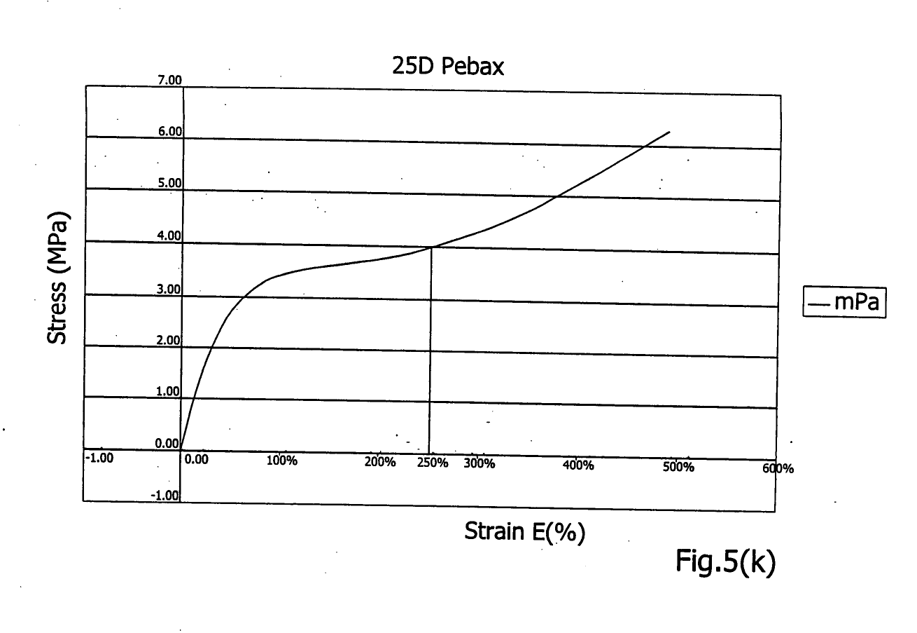

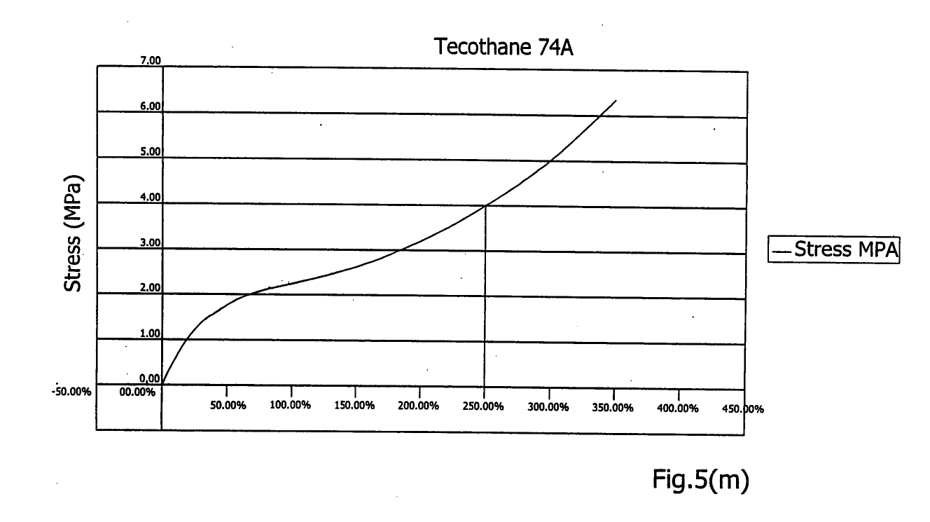

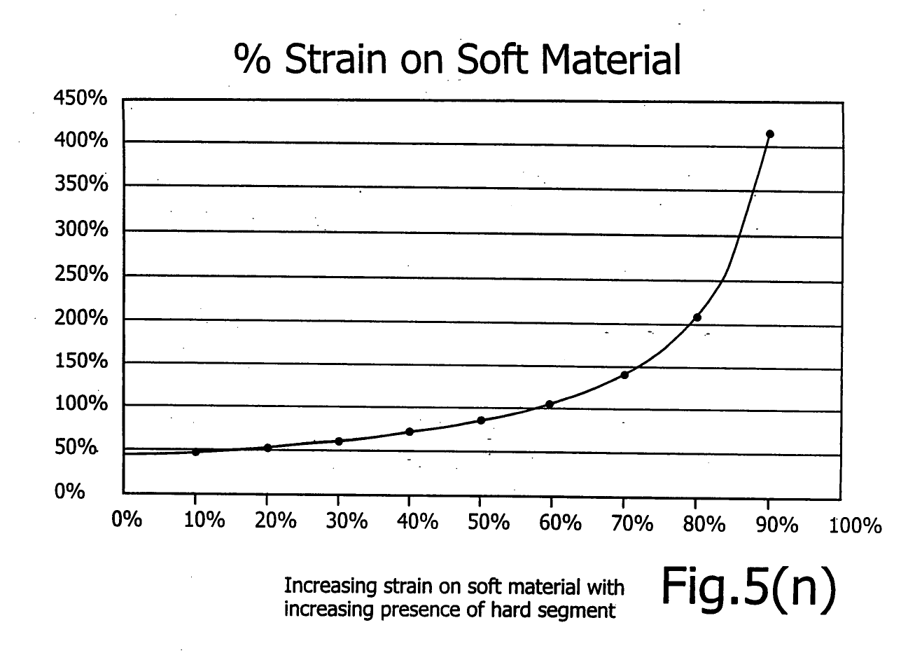

It has been found that the resistance of the retrieval catheter tip 1 to buckling is maximised by providing two reinforcement columns 3. During analysis of the tip 1, it was observed that the tip 1 having two reinforcement columns 3 (FIG. 5(a)) provided a greater level of buckle resistance compared to, for example, a tip 11 having four reinforcement columns 3 (FIG. 5(b)) but with the same overall column cross-sectional area. It is believed that this effect arises due to the effective area A₁ of the columns 3 in FIG. 5(a) being greater than the combined effective areas A₁₁ of the columns 3 in FIG. 5(b). The columns 3 act like a beam to prevent buckling, and thus the larger effective area A₁ of the tip 1 of FIG. 5(a) provides greater reinforcement than the smaller combined effective areas A₁₁ of the tip 11 of FIG. 5(b).

Also the point of collapse of the tip 1 with two reinforcements 3 is located further proximally of the distal end compared to a tip having three or four reinforcement columns 3. This enables the tip 1 to be of a greater length.

In addition, it was observed that the resistance to buckling of the retrieval catheter tip 1 is maximised by extending each reinforcement column 3 as far around the circumference of the tip 1 as possible while providing sufficient tip body 2 to facilitate expansion. As illustrated in FIGS. 5(c) and 5(d), the tip 1 of FIG. 5(c) provides a greater level of buckle resistance compared to, for example, a tip 12 having the same overall cross-sectional area of columns 3 but with thicker columns 3 that extend around less of the tip circumference (FIG. 5(d)). It is believed that this effect arises due to the effective area A₁ of the columns 3 in FIG. 5(c) being greater than the effective area A₁₂ of the columns 3 in FIG. 5(d), and thus the larger effective area A₁ of the tip 1 of FIG. 5(c) provides greater reinforcement than the smaller effective area A₁₂ of the tip 12 of FIG. 5(d).

By extending the arcuate columns 3 around a sufficient part of the circumference of the tip, this results in a higher second moment of area of the columns 3. This is because an extension in the circumferential dimension of the column 3 results in an increase in both the height and the width of the column effective area A₁, as illustrated in FIG. 5(a).

The resultant column 3 is substantially “C” shaped. This “C” shaped column 3 will better resist buckling than a simple round or rectangular cross-section element. This is because the curved or circumferential nature of the columns 3 results in an effective area A₁ greater than the simple cross-sectional area of the columns 3.

As illustrated in FIG. 5(c), the wall thickness of the columns 3 is greater than the wall thickness of the tip body 2. This aspect of the invention provides significant operational advantages. The thin-walled tip body 2 is more easily expandable to accommodate relatively large articles, such as an embolic protection filter with a large load of retained emboli, during retrieval. In combination with this, the thick-walled columns 3 have greater buckle resistance, and thus the overall buckle resistance of the retrieval catheter tip 1 is maximised.

These operational advantages of enhanced expandability of the tip body 2 and enhanced buckle resistance of the column 3 can also be achieved by configuring the tip such that the ratio of the cross-sectional area of the column 3 to the circumferential dimension of the column 3 is greater than the ratio of the cross-sectional area of the tip body 2 to the circumferential dimension of the tip body 2.

In the retrieval catheter tip of the invention, the ratio of the wall thickness of the column to the wall thickness of the tip body is preferably in the range from 1 to 10. For example, in the tip of FIG. 5(e), the wall thickness 2t of the columns 3 is twice the wall thickness t of the tip body 2. In FIG. 5(f) the wall thickness 3t of the columns 3 is three times the wall thickness t of the tip body 2, in FIG. 5(g) the wall thickness 4t of the columns 3 is four times the wall thickness t of the tip body 2, and in FIG. 5(h) the wall thickness St of the columns 3 is five times the wall thickness t of the tip body 2.

By increasing the wall thickness of the columns 3 and maintaining the wall thickness of the tip body constant, a large ratio of wall thickness may be achieved while maintaining catheter structural integrity.

Alternatively the wall thickness of the columns 3 may be maintained constant and the wall thickness of the tip body 2 decreased. For example in FIG. 5(i) the wall thickness 2t of the columns 3 is five times the wall thickness 0.4t of the tip body 2, and in FIG. 50) the wall thickness 2t of the columns 3 is ten times the wall thickness 0.2t of the tip body 2.

By decreasing the wall thickness of the tip body and maintaining the wall thickness of the columns constant, a large ratio of wall thickness may be achieved while minimising the overall crossing profile of the tip. In addition, easier expansion of the tip may be achieved with the tip body of decreased wall thickness. For these reasons, it is preferred to decrease the wall thickness of the tip body rather than to increase the wall thickness of the columns to achieve the desired ratio of wall thickness.

It has been found that the preferred ratio of wall thickness is for the wall thickness of the columns to be twice the wall thickness of the tip body.

By way of example, a retrieval catheter tip having two columns 3 of Pellethane 75D and a tip body 2 of Tecothane 74A was tested.

As illustrated in FIG. 5(m), the stress-strain curves for Tecothane 74A has a point of inflection at a strain of approximately 250%. At strains above this value, the forces required to expand the tip body 2 increases at a greater rate. Thus it is preferable to operate with a strain on the tip body 2 safely below 250%.

In this example, the tip was designed to expand by 200% upon retrieval of an article into the tip. FIG. 5(n) illustrates the variation for such a tip of percentage strain on the tip body 2 (y-axis) with the percentage of the columns 3 in the circumference of the tip (x-axis).

To maintain the strain on the tip body 2 safely below 250%, the percentage of the columns 3 in the tip should be chosen to be less than 80%. To obtain a tip with sufficient buckle resistance, the percentage of the columns 3 in the tip should be chosen to be between 60% and 70%. Thus the tip is ideally configured with between 60% and 80% of columns 3 in the tip to achieve both expandability and buckle resistance. These percentages correspond approximately to an angular extension of each reinforcement column 3 around the circumference of the tip being in the range of from 105 degrees to 145 degrees. In the particularly preferred arrangement illustrated in FIGS. 5(a) and 5(d), the angular extension of each reinforcement column 3 around the circumference of the tip is approximately 126 degrees, which corresponds approximately to 70% of the tip being of the columns 3.

An alternative material for the tip body 2 is Pebax 25D. as illustrated in FIG. 5(k), the stress-strain curve for Pebax 25D also has a point of inflection at a strain of approximately 250%.

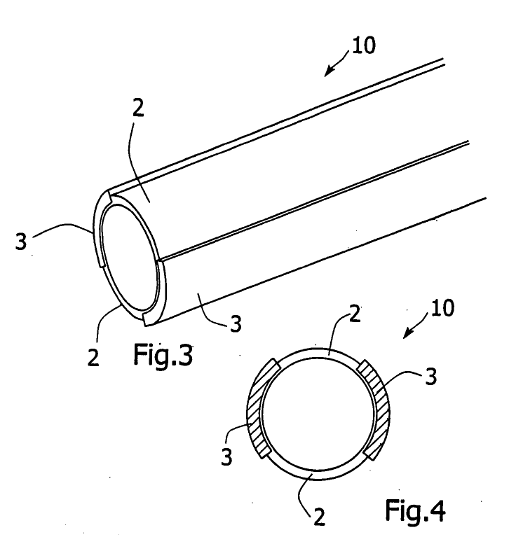

The tip may comprise means to ease passage of the tip and/or to ease passage of an article into the tip. For example, the columns 3 may be partially embedded within the tip body 2, and the columns 3 may extend radially outwardly of the tip body 2 to define two longitudinally extending protrusions, as illustrated in the catheter 10 of FIGS. 3 and 4. The protruding columns 3 have a lower coefficient of friction than the tip body 2. Thus, the protruding columns 3 define a low coefficient of friction track to ease passage of the retrieval catheter tip 10 through a body passageway of a patient.

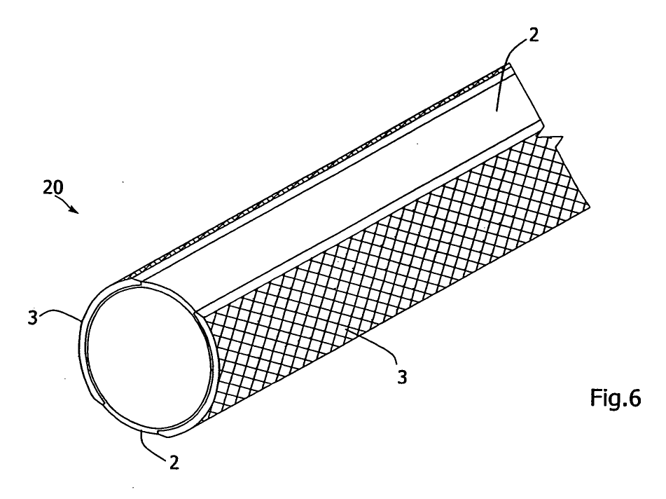

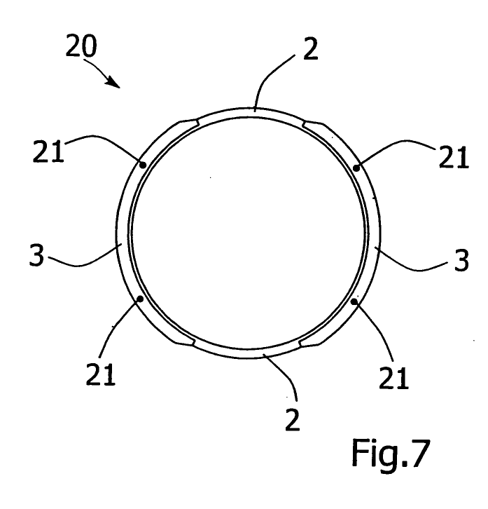

FIGS. 6 and 7 illustrate another retrieval catheter tip 20 according to the invention, which is similar to the tip 10 of FIGS. 3 and 4, and similar elements in FIGS. 6 and 7 are assigned the same reference numerals.

In this case, the columns 3 are of a composite material having wire reinforcements 21 embedded therein. The composite construction of the columns 3 enhances the buckle resistance of the tip 20.

It will be appreciated that other forms of reinforcement may be provided in addition to or instead of wire reinforcements, such as fibre reinforcements, and/or particle reinforcements.

The tip body 2 could alternatively or additionally be of a composite material. In one case when the entire tip is of a composite material, it may be configured to expand when a compressive load is applied to it, for example during retrieval of an embolic protection filter into the tip.

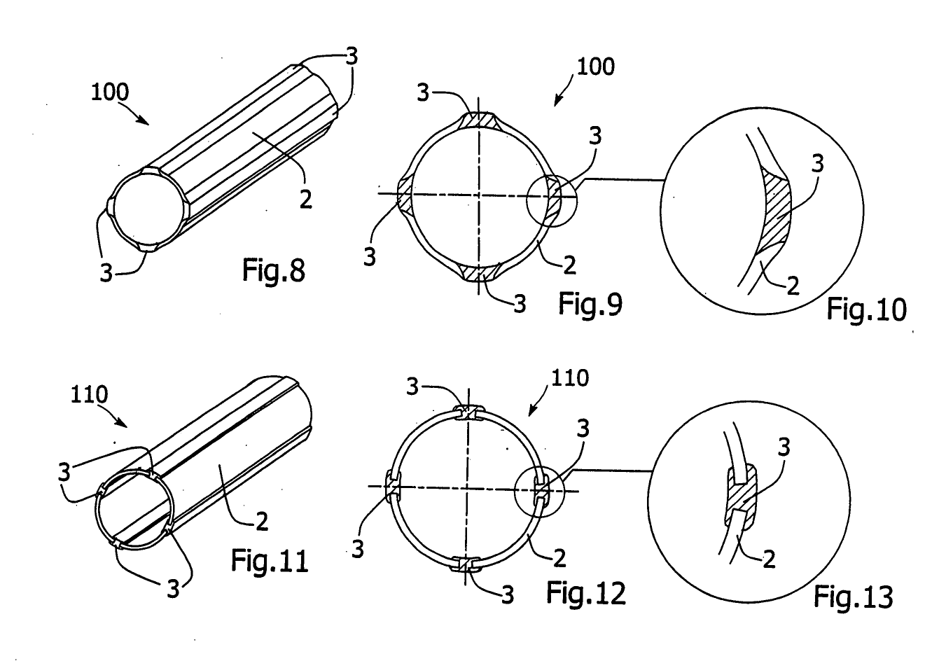

Referring to FIGS. 8 to 10 there is illustrated another retrieval catheter tip 100 according to the invention, which is similar to the tip 10 of FIGS. 3 and 4, and the same reference numerals are assigned to similar elements in FIGS. 8 to 10.

The tip 100 comprises four columns 3 equi-spaced circumferentially around the tip body 2. The columns 3 extend radially outwardly of the tip body 2 to define four longitudinally extending protrusions (FIG. 8). The protruding columns 3 define a low coefficient of friction track to ease passage of the tip 100 through a body passageway, such as a vasculature.

FIGS. 11 to 13 illustrate another retrieval catheter tip 110 according to the invention, which is similar to the tip 100 of FIGS. 8 to 10, and similar elements in FIGS. 11 to 13 are assigned the same reference numerals.

In this case, the columns 3 extend both radially outwardly and radially inwardly of the tip body 2 to define four longitudinally extending protrusions (FIGS. 11 and 12). Thus, the protruding columns 3 define a low coefficient of friction track to ease passage of the retrieval catheter tip 110 through the body of a patient, and also to ease passage of an article into the tip 110, for example during retrieval of an embolic protection filter into the tip 110.

The cross section of the columns 3 is substantially I-shaped (FIG. 13) to ensure a high second moment of area, and thus minimise the possibility of buckling of the tip 110. In addition, the I-shaped cross section of the columns 3 defines a large surface area between the tip body 2 and the columns 3 for a secure bonding of the tip body 2 with the columns 3.

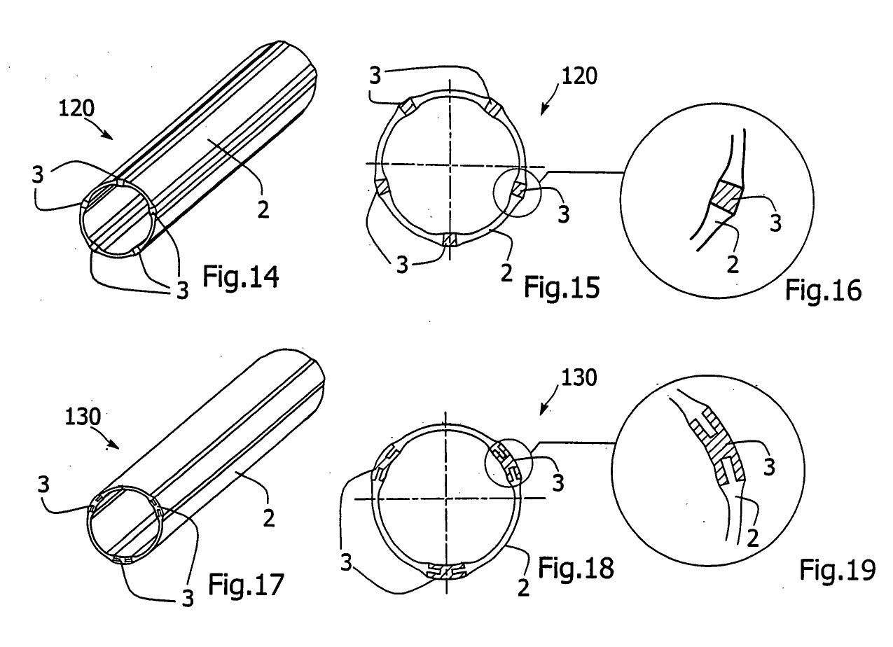

Referring next to FIGS. 14 to 16 there is illustrated another retrieval catheter tip 120 according to the invention, which is similar to the tip 100 of FIGS. 8 to 10, and the same reference numerals are assigned to similar elements in FIGS. 14 to 16.

In this case, there are five reinforcement columns 3 which extend radially outwardly and radially inwardly of the tip body 2, and are equi-spaced apart circumferentially around the tip body 2 (FIG. 15). The columns 3 are substantially trapezoidal in cross-section, and act to prevent circumferential buckling of the tip 120.

Referring to FIGS. 17 to 19 there is illustrated another retrieval catheter tip 130 according to the invention, which is similar to the tip 100 of FIGS. 8 to 10, and the same reference numerals are assigned to similar elements in FIGS. 17 to 19.

In this case, three reinforcement columns 3 are provided extending radially outwardly and radially inwardly of the tip body 2, and equi-spaced apart circumferentially around the tip body 2 (FIG. 18). By providing only three reinforcement columns 3 the cross sectional area of the columns 3 relative to the cross sectional area of the tip body 2 is reduced for increased radial expansion of the tip 1.

The cross section of the columns 3 is substantially “I” shaped (FIG. 18) to minimise the possibility of buckling of the tip 130, and also to ensure secure bonding of the tip body 2 with the columns 3.

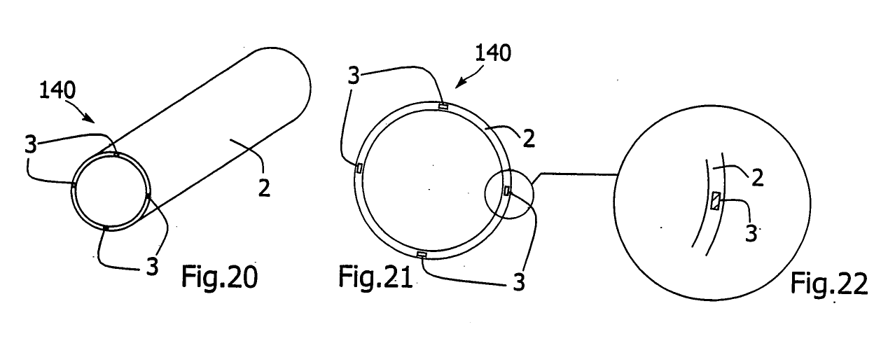

Referring to FIGS. 20 to 22 there is illustrated another retrieval catheter tip 140 according to the invention, which is similar to the tip 100 of FIGS. 8 to 10, and the same reference numerals are assigned to similar elements in FIGS. 20 to 22.

In this case, the reinforcement columns 3 are completely embedded within the tip body 2 (FIG. 21) to prevent delamination between the columns 3 and the tip body 2.

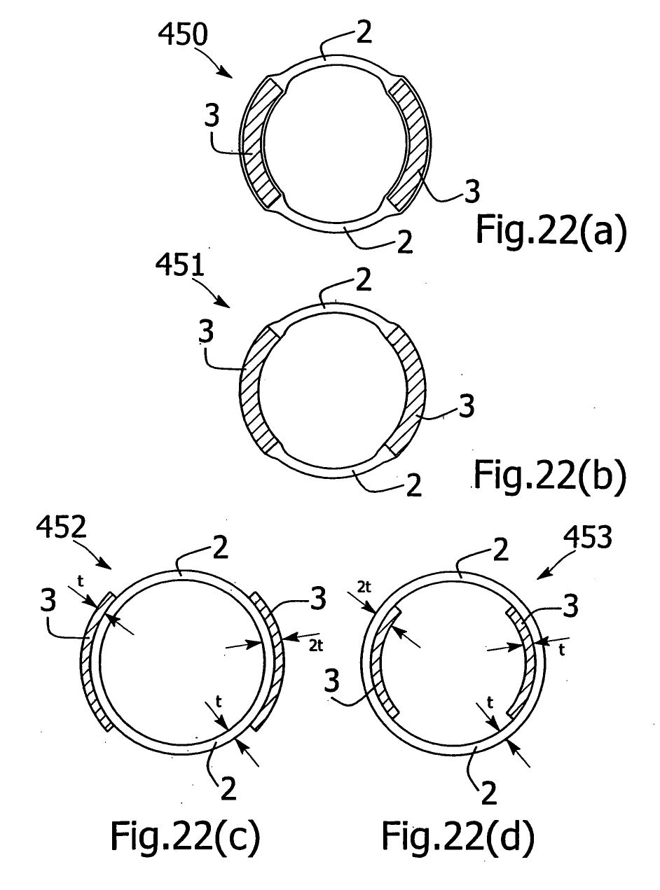

Another retrieval catheter tip 450 according to the invention is illustrated in FIG. 22(a). The columns 3 are arcuate and extend over a substantial circumferential dimension of the tip 450 for enhanced buckling resistance. Also the columns 3 are completely embedded within the tip body 2 for a secure adhesion between the tip body 2 and the columns 3.

As illustrated in the tip 451 of FIG. 22(b), the two wings of the tip body material may be completely separated by the columns 3. There are thus no thin layers of tip body material along the inner surface of the columns 3 in contrast to the tip 1 of FIG. 2.

The columns 3 may alternatively be provided on an external surface of the tip body 2, as illustrated in the tip 452 of FIG. 22(c), or be provided on an internal surface of the tip body 2 as illustrated in the tip 453 of FIG. 22(d). Any suitable means may be used, such as bonding, to fix the columns 3 to the tip body 2.

With regard to the ratio of the thickness of the component parts of the tip 452 of FIG. 22(c), the wall thickness of the columns 3 is t and the thickness of the tip body 2 is t which results in an overall thickness of 2t for the tip 452 at the location of the columns 3. Thus in the case of the tip 452, the ratio of the overall wall thickness 2t of the tip 452 at the location of the columns 3 to the wall thickness t of the tip body 2 is two for enhanced buckle resistance.

Similarly in the case of the tip 453 of FIG. 22(d), the overall thickness 2t of the tip 453 at the location of the columns 3 is twice the wall thickness t of the tip body 2.

The cross-sectional area of the tip body 2, or the cross-sectional area of the reinforcement columns 3, or the overall cross-sectional area of the tip may vary along the length of the tip. However, in all retrieval catheter tips of the invention, the reinforcement columns 3 are configured to provide substantially uniform reinforcement to the tip along the length of the tip. One means of achieving this uniform reinforcement is to maintain the ratio of the circumferential dimension of the reinforcements 3 to the circumferential dimension of the tip body 2 constant along the length of the tip. When calculating the circumferential dimension of the tip body 2, the two thin layers 4 of tip body material illustrated in FIG. 2 are disregarded, and only the circumferential dimension of the large wings of the tip body 2 are taken into account.

By maintaining this circumferential dimension ratio constant, variations in the various cross-sectional areas may occur while maintaining the reinforcement uniform along the length of the tip.

For example, the inner diameter of the tip body 2 may remain constant while the outer diameter decreases distally along the length of the tip 150, as illustrated in FIG. 24, such that the tip 150 tapers distally inwardly. The distally decreasing outer diameter of the tip 150 provides for a smooth transition through a vasculature. The tip 150 may be formed by drawing the tip 150 while heating. Typically, the tip 150 would be drawn down over a mandrel.

Alternatively the outer diameter of the tip body 2 may remain constant while the inner diameter increases distally along the length of the tip 160, as illustrated in FIG. 25, such that the tip 160 tapers distally outwardly. With this configuration less expansion of the tip 160 is required to retrieve an article into the tip. Thus the overall retrieval forces are reduced.

The outwardly tapering tip 160 of FIG. 25 may however be more prone to snagging on a stent on the like.

The outer diameter of the tip body 2 may remain constant over a proximal part 171 of the tip 170 and decrease distally in a linear manner over a distal part 172 of the tip 170, as illustrated in FIG. 26, such that the tip 170 tapers distally inwardly. In this manner, the tip 170 of FIG. 26 enjoys more column support closer to a distal end of the tip 170 as compared with the tip 150 of FIG. 24.

Alternatively the outer diameter of the tip body 2 may decrease distally in a non-linear manner over an intermediate part 181 of the tip 180, with a constant diameter proximal part 182 and a constant diameter distal part 183 of the tip 180, as illustrated in FIG. 27.

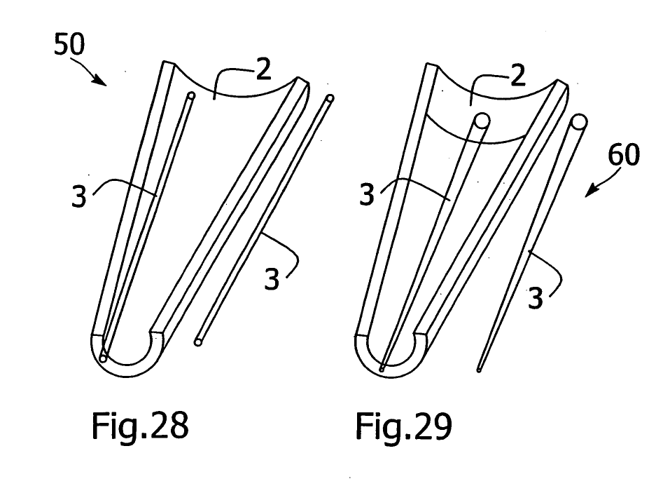

In the case of the tip 50 of FIG. 28, the tip 50 is tapered distally inwardly and has a substantially constant cross-sectional area along the length of the tip 50.

In the tip 60 of FIG. 29, the tip 60 is tapered distally inwardly and has a cross-sectional area that decreases distally along the length of the tip 60. The ratio of the cross-sectional area of the tip body 2 to the cross-sectional area of the columns 3 is constant along the length of the tip 60. This provides good transition, good tip expansion characteristics and also provides compressive resistance.

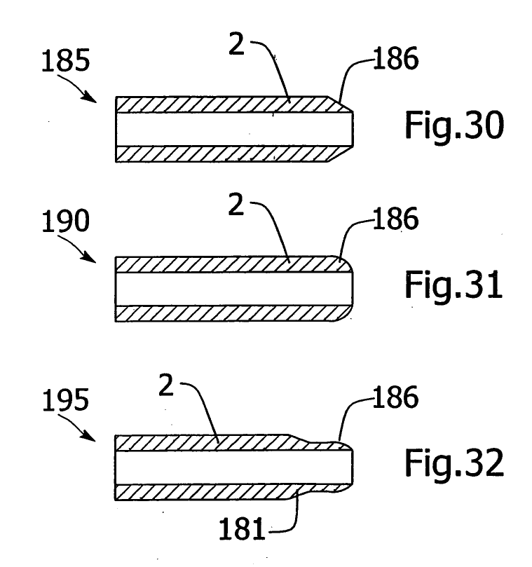

FIG. 30 illustrates another retrieval catheter tip 185 according to the invention, which is similar to the tip 1 of FIG. 23. In this case, the tip body 2 tapers distally inwardly at the distal end 186 to present a low-profile leading edge and thus enable atraumatic advancement of the tip 185 through a body passageway, such as a vasculature, and to minimise the possibility of snagging of the tip 185 during advancement, for example on a medical device, such as a stent.

Alternatively the tip body 2 may be rounded at the distal end 186 to provide a tip 190 with a smooth crossing profile, as illustrated in FIG. 31.

The tip body 2 of FIG. 32 has a rounded distal end 186 and an outer diameter that decreases distally in a non-linear manner over an intermediate part 181 of the tip 195.

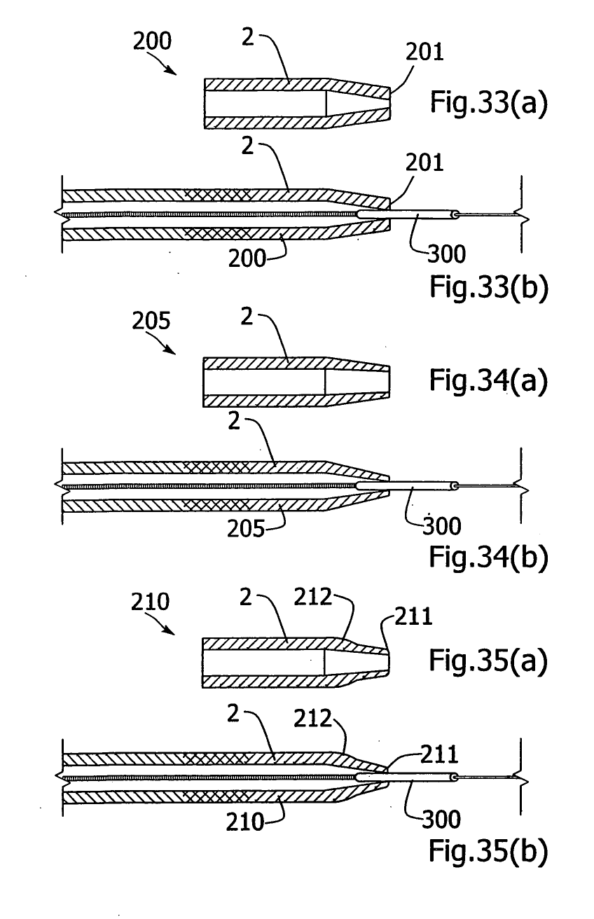

Referring to FIGS. 33(a) and (b), there is illustrated another retrieval catheter tip 200 according to the invention, which is similar to the tip 1 of FIG. 23.

The distal end 201 of the tip body 2 tapers distally inwardly (FIG. 33(a)). In this manner a close interference fit may be achieved between the tip 200 and a centring catheter 300 protruding distally out of the tip 200 (FIG. 33(b)). By achieving such an interference fit, a smooth transition between the centring catheter 300 and the tip 200 may be achieved to prevent snagging of a stent or the like on the distal end 201 of the tip 200 during advancement through a body passageway, such as a vasculature.

In the case of the tip 200 of FIGS. 33(a) and (b), the wall thickness of the tip body 2 remains constant along the length of the tip 200.

To facilitate retrieval of an article, such as an embolic protection filter, into the tip 200, the centring catheter 300 is withdrawn proximally relative to the tip 200. The centring catheter 300 will then provide a degree of support to the tip 200 at a point proximally of the distal end 201.

Alternatively the wall thickness of the tip body 2 may decrease distally along the length of the tip 205 in a linear manner, as illustrated in FIG. 34(a). A close interference fit between the distally inwardly tapering tip 205 and the centring catheter 300 is achieved in use (FIG. 34(b)).

In the tip 210 of FIGS. 35(a) and (b), the distal end 211 of the tip body 2 is rounded for a smooth crossing profile. The distal end 211 also tapers distally inwardly for snag-free advancement through a body passageway (FIG. 35(b)). Furthermore, the wall thickness of the tip body 2 decreases distally in a non-linear manner over an intermediate portion 212 of the tip 210 (FIG. 35(a)).

In FIGS. 24 to 35(b), the wall thickness of the tip body 2 has been described and illustrated as varying along at least part of the length of the retrieval catheter tip, and the tip body 2 has been described and illustrated as tapering along at least part of the length of the retrieval catheter tip. However, it will be understood that the wall thickness of the reinforcement columns 3 could alternatively or additionally vary along at least part of the length of the tip. In addition, the columns 3 may alternatively or additionally taper along at least part of the length of the retrieval catheter tip. Indeed, a variety of combinations of wall thickness/tapering of the tip body 2 and wall thickness/tapering of the reinforcement columns 3 are possible. For example, in one retrieval catheter tip the wall thickness of the tip body 2 may vary as illustrated in FIG. 26, and the reinforcement columns 3 may be of constant wall thickness as illustrated in FIG. 23.

A particularly preferred combination is a retrieval catheter tip having reinforcement columns 3 with a tapered or rounded distal end 186, as illustrated in FIG. 30 or 31, and a tip body 2 tapering distally as illustrated in FIGS. 24, 25, 26 or 30. This combination is preferred because the columns 3 provide support along all of the tip to the distal end, and the tip body 2 is thinner at the distal end for enhanced expandability.

It will be appreciated that a wide variety of possible configurations for the tip are possible within the scope of the invention.

The tip body material and the column material may be selected from different groups of materials, in which case the materials are selected to ensure that the tip body 2 and the columns 3 adequately adhere to one another and have suitable relative hardness characteristics. Adhesion between the columns 3 and the tip body 2 may be achieved by means such as chemical or mechanical means, and preferably by thermal/pressure induced adhesion. Further suitable mechanical adhesion means include notching, surface roughness, encapsulation, keying, or any suitable combination of the aforementioned adhesion means.

By selecting the tip body material and the column material from different groups of materials, a large difference in the modulus of elasticity of the columns 3 relative to the tip body 2 may be obtained. In this manner the columns 3 may be chosen from a high modulus of elasticity material, such as stainless steel, without requiring a high modulus of elasticity for the tip body 2. By selecting a high modulus of elasticity column material, the cross-sectional area of the columns 3 can be minimised while maintaining sufficient compressive strength. In this way, the cross-sectional area of the tip body 2 relative to the cross-sectional area of the columns 3 can be maximised to facilitate radial expansion of the tip for retrieving an article into the tip.

The invention provides a retrieval catheter tip with at least one reinforcement 3 column extending along the tip body 2. By ensuring sufficient bond strength between the soft tip body 2 and the columns 3, by minimising the wall thickness of the tip body 2, and by maximising the circumference of the tip body 2, increased radial expansion of the tip may be achieved for a low retrieval force. In this manner, the column strength to prevent buckling is minimised and the cross-sectional area of the columns 3 can be reduced. Thus the invention provides a low profile, thin-walled, highly expansible tip. The tip facilitates expansion upon retrieval of an article into the tip, and also maintains sufficient column strength to prevent buckling of the tip.

The cross-sectional area of the reinforcement column may or may not change relative to the cross-sectional area of the tip body along the length of the retrieval catheter tip. The overall cross-sectional area of the tip also may or may not change along the length of the tip This ensures a high expansion capability at the distal tip and also along the length of the tip. In this way the likelihood of buckling of the tip during retrieval of an article into the tip is reduced.

A taper can be provided over the distal section of the tip by reducing the diameter at the distal end of the tip. This prevents snagging of the tip during advancement through a tortuous anatomy or a medical device, such as a stent.

The number of reinforcement columns and the column strength of each column can be selectively altered, and similarly the flexibility of the tip can be selectively altered by means of the co-extrusion process to form a retrieval catheter tip with the desired expansion and buckling-resistant characteristics.

In addition, co-extrusion onto a mandrel facilitates accurate sizing of the tip. The bond strength between the tip body and the reinforcement columns can be enhanced by co-extrusion under pressure.

The low coefficient of friction protrusions facilitate rapid and easy passage of the retrieval catheter tip, and also facilitate rapid and easy passage of an article into the tip. In this way, there is less possibility of buckling of the retrieval catheter tip upon retrieval of an article into the tip, or upon advancement of the retrieval catheter tip through the body of a patient.

The reinforcement columns 3 extend along the tip body for a length in the range of from 3 mm to 20 mm, and preferably for a length of approximately 13 mm. It has been found that with reinforcement columns 3 of such a length, the tip effectively resists buckling during retrieval of an article into the tip. Also a tip of approximately 13 mm in length has an adequate active retrieval area for an embolic protection filter of approximately 20 mm or less in length.

The reinforcement columns 3 may extend proximally over at least part of the retrieval catheter shaft 401.

It will be appreciated that other means of forming the retrieval catheter tip of the invention other than co-extrusion are possible.

For example, the tip body may be extruded and the reinforcement column then bonded to the internal/external surface of the tip body to form the tip. However this would be a relatively expensive forming method, and achieving a secure bond between the column and the tip body may be difficult.

Alternatively injection moulding could be used to form the tip. This is also an expensive forming method, and achieving a secure bond between the column and the tip body may be difficult.

Means to facilitate visualisation of the retrieval catheter tip could also be provided. For example a filler of radiopaque material, such as tungsten, could be included in the tip.

The invention is not limited to the embodiments hereinbefore described, with reference to the accompanying drawings, which may be varied in construction and detail.

1. A retrieval catheter tip for retrieving an article into the tip, the tip comprising:—
an expandable tip body for retrieving an article into the tip;
the tip body being resilient to retain a retrieved article within the tip; and
a reinforcement column extending along the tip body;
the column being configured to provide substantially uniform reinforcement to the tip substantially along the length of the column;
a ratio of a circumferential dimension of the reinforcement column to a circumferential dimension of the tip body being substantially constant along the length of the tip, and
the external surface of the tip body being substantially smooth.

2. (canceled)

3. A tip as claimed in claim 1 wherein the ratio of the wall thickness of the column to the wall thickness of the tip body is substantially constant along the length of the tip.

4. A tip as claimed in claim 1 wherein the ratio of the cross-sectional area of the column to the cross-sectional area of the tip body is substantially constant along the length of the tip.

5. A tip as claimed in claim 1 wherein the column is substantially arcuate in cross-section.

6. A tip as claimed in claim 5 wherein the angular extension of the column around the circumference of the tip is in the range of from 10 degrees to 145 degrees.

7. A tip as claimed in claim 6 wherein the angular extension of the column around the circumference of the tip is in the range of from 105 degrees to 145 degrees.

8. A tip as claimed in claim 7 wherein the angular extension of the column around the circumference of the tip is approximately 126 degrees.

9. A tip as claimed in claim 1 wherein the wall thickness of the tip at the location of the column is greater than or equal to the wall thickness of the tip body.

10. A tip as claimed in claim 9 wherein the wall thickness of the tip at the location of the column is between 1 and 10 times the wall thickness of the tip body.

11. A tip as claimed in claim 10 wherein the wall thickness of the tip at the location of the column is two times the wall thickness of the tip body.

12. A tip as claimed in claim 1 wherein the ratio of the cross-sectional area of the column to the circumferential dimension of the column is greater than or equal to the ratio of the cross-sectional area of the tip body to the circumferential dimension of the tip body.

13. A tip as claimed in claim 12 wherein the ratio of the cross-sectional area of the column to the circumferential dimension of the column is between 1 and 10 times the ratio of the cross-sectional area of the tip body to the circumferential dimension of the tip body.

14. A tip as claimed in claim 13 wherein the ratio of the cross-sectional area of the column to the circumferential dimension of the column is two times the ratio of the cross-sectional area of the tip body to the circumferential dimension of the tip body.

15. A tip as claimed in claim 1 wherein the column extends along substantially the full length of the tip body.

16. (canceled)

17. (canceled)

18. A tip as claimed in claim 1 wherein the tip is of a length in the range of from 3 mm to 20 mm.

19. A tip as claimed in claim 18 wherein the tip is of a length of approximately 13 mm.

20. A tip as claimed in claim 1 wherein the tip at least partially tapers distally inwardly.

21. (canceled)

22. A tip as claimed in claim 1 wherein the wall thickness of the column and/or of the tip body varies along at least part of the length of the tip.

23. A tip as claimed in claim 1 wherein the wall thickness of the column and/or of the tip body is substantially constant along at least part of the length of the tip.

24. A tip as claimed in claim 1 wherein the tip is shaped for a smooth crossing profile.

25. (canceled)

26. A tip as claimed in claim 24 wherein a distal end of the tip tapers distally inwardly.

27. A tip as claimed in claim 1 wherein the tip comprises two or more columns spaced apart circumferentially around the tip.

28. A tip as claimed in claim 27 wherein the tip comprises two columns.

29. A tip as claimed in claim 27 wherein the columns are substantially equi-spaced apart.

30. A tip as claimed in claim 1 wherein the tip body is integrally formed with the column to form the tip.

31. A tip as claimed in claim 30 wherein the tip body and the column are co-extruded to form the tip.

32. (canceled)

33. (canceled)

34. (canceled)

35. A tip as claimed in claim 1 wherein the column is provided at least partially on an external and/or internal surface of the tip body.

36. (canceled)

37. (canceled)

38. (canceled)

39. A tip as claimed in claim 1 wherein the column and the tip body are at least partially of materials selected from the same groups of materials.

40. A tip as claimed in claim 39 wherein the column and the tip body are at least partially of materials selected from the polyurethane group of materials.

41. A tip as claimed in claim 40 wherein the column is of a material with a higher isocyanate concentration than the material of the tip body.

42. (canceled)

43. (canceled)

44. (canceled)

45. (canceled)

46. (canceled)

47. (canceled)

48. (canceled)

49. (canceled)

50. (canceled)

51. A tip as claimed in claim 1 wherein the tip comprises a hydrophilic coating.

52. (canceled)

53. (canceled)

54. (canceled)

55. (canceled)

56. (canceled)

57. (canceled)

58. A tip as claimed in claim 1 wherein the tip comprises a low friction coating.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. A retrieval catheter tip for retrieving an article into the tip, the tip comprising:—
an expandable tip body for retrieving an article into the tip;
the tip body being resilient to retain a retrieved article within the tip; and
a reinforcement column extending along the tip body;
a ratio of a circumferential dimension of the reinforcement column to a circumferential dimension of the tip body being substantially constant along the length of the tip, and
the internal surface of the tip body being substantially smooth.

81. A retrieval catheter tip for retrieving an article into the tip, the tip comprising:—
an expandable tip body for retrieving an article into the tip;
the tip body being resilient to retain a retrieved article within the tip; and
a reinforcement column extending along the tip body;
a ratio of a circumferential dimension of the reinforcement column to a circumferential dimension of the tip body being substantially constant along the length of the tip, and
the external surface of the tip body being substantially continuous.

82. A retrieval catheter tip for retrieving an article into the tip, the tip comprising:—
an expandable tip body for retrieving an article into the tip;
the tip body being resilient to retain a retrieved article within the tip; and
a reinforcement column extending along the tip body;
a ratio of a circumferential dimension of the reinforcement column to a circumferential dimension of the tip body being substantially constant along the length of the tip, and
the internal surface of the tip body being substantially continuous.
